(19) United States
(12) Patent Application Publication
(10) Pub. No.: US 2003/0014002 A1
(43) Pub. Date: Jan. 16, 2003

(54) Wrist support for providing protection against carpal tunnel stress

(76) Inventor: Lilian Quintana, San Francisco, CA (US)

Correspondence Address: Melvin K. Silverman, SUITE 440, 4901 NORTH FEDERAL HIGHWAY, FORT LAUDERDALE, FL, 33308, US

(21) Appl. No.: 10/228,314

(22) Filed: Aug. 26, 2002

Related U.S. Application Data
(63) Continuation-in-part of application No. 09/642,603, filed on Aug. 22, 2000.

(51) Int. Cl. A61F 5/00; A61F 13/00

(52) U.S. Cl. 602/64; 602/21

(57) ABSTRACT

A removable wrist cuff provides anatomical support for the relief of carpal tunnel stress upon the wrist and forearms with respect to repetitive motions of the hands and fingers. The wrist support includes a circumferential fabric device for the encirclement of a user's wrist and which is detachable from the wrist by the use of hook-and-loop fasteners at opposite and opposing ends. The bottom or palmar side of the cuff is concave or crescent shaped in radial transverse cross-section such that the radial dimension between inner and outer axial surfaces tapers symmetrically at either side of the base of the wrist such that the top part of the cuff, when secured to the wrist, is composed of fabric only, this inclusive of the fasteners at opposing surfaces of ends of the cuff. The palmer side is preferably formed of a light, high-density foam, secured between opposing axial surfaces of wrist enveloping surfaces which, in radial cross-section, define the crescent-like geometry. This shape, defined by a light, high-density foam, provides suitable support with respect both to pressure and protection against vibration, at the underside of the wrist where it is anatomically most important to provide such protection. The thickness of the high-density foam, at the vertical axis of the device, is approximately equal to its axial width.

### REFERENCE TO RELATED APPLICATION

This case is a continuation-in-part of application Ser. No. 09/642,603, filed Aug. 22, 2000.

Not Applicable.

### BACKGROUND OF THE INVENTION

Increasing numbers of individuals now spend a substantial part of their time, whether at home or office, in the carrying out of tasks which involve repetitive motions of the hand and fingers, for example, typing upon a computer keyboard, sewing, and certain types of industrial assembly line movements. As is known, excessive time spent in such repetitive motions can lead to pain and stiffness of the wrists, arms and even the shoulders. Individuals that carry out such tasks on a daily basis can develop a condition known as carpal tunnel syndrome, and this condition has become recognized as one of the most common workplace related injuries. Further, carpal tunnel syndrome can be debilitating and, at least, can cause loss of time at work. In a worst case, it can lead to permanent disability. Accordingly, there has developed a need in the art for a light, streamlined, comfortable, and inexpensive wrist support that does not interfere with one's clothing and which does not otherwise interfere with normal anatomic motions associated with tasks carried out upon a keyboard, sewing machine, or industrial assembly line, and which does not require removal when tasks apart from the keyboard or the like, such as going to the restroom, are required. The instant invention thereby responds to these needs while providing essential physical support to the wrist to thereby reduce carpal tunnel associated stress.

The problem of carpal tunnel stress and its associated syndrome has been recognized in the art, particularly with respect to computer related equipment such as the keyboard and mouse. Efforts of the prior art to address this problem fall basically into two categories, namely, either modifications or enhancements to the keyboard itself or a device which can be worn up on the hand, fingers or wrist of the user to typically elevate the wrist relative to the keyboard and to provide protection against vibration therefrom.

Representative patents show integration of elements into a computer keyboard for the protection of the wrist during use thereof are represented by U.S. Pat. No. 5,234,186 (1993) to Powell, entitled Wrist Rest for Keyboard and No. 5,730,403 (1998) to Johnston, entitled Arched Panel Wrist Support. Patents which show integration of a wrist and/or arm support into a mouse pad are represented by U.S. Pat. No. 5,335,828 (1994) to Thomsen, entitled Four Arm Support Cradle, and No. 5,918,840 (1999) to Christensen, entitled Integrated Mouse Pad and Wrist and Arm Support.

With respect to devices, independent of a keyboard or mouse, intended to protect against carpal tunnel syndrome, are structures, which typically wrap around the lower base of the hand and around the wrist. Such approaches are reflected in U.S. Pat. Nos. 5,439,192 (1995) to King, entitled Hand-Worn Device For Elevation The Hand And Fingers With Respect To A Keyboard; and No. 5,925,007 (1999) to Ashline, entitled Carpal Cuff.

Another approach involves the use of pads or the like. Design Patent No. 415,480 (1999) to Kendall, et al, entitled Combined Wrist And Hand Support relates to a circular stand alone pad which is simply positioned underneath the wrist of the user but which has no physical attachment thereto. Closely related to Kendall is U.S. Pat. No. 5,348,408 (1994) to Gerlardi, et al, entitled Wrist Pad For Use With Keyboards. The only prior art known to the within Inventor that teaches a wrist support device in the nature of a compressible material physically attached onto or about the wrist of the user is reflected in PCT published specification WO 99/05062 (1999) to Beh, et al, entitled Portable Wrist Support Pad For Use With Computer Input Devices. The instant invention may, accordingly, be viewed as a direct improvement over the cumbersome structure of Beh and limited solutions of Kendall and Garlardi.

### SUMMARY OF THE INVENTION

The instant invention relates to a removable wrist cuff to provide anatomical support to, and protection of, the wrist during repetitive motions of the hand and fingers, said wrist cuff comprises an inner circumferential strap proportioned in axial width, circumferential length and circumferential curvature for securement about the entirety of an adult human wrist; a solid body of a compressibly resilient material, of substantially like axial width of said inner strap, an anterior surface of said body adhered to a palmer outer surface of said inner strap, said solid body of resilient material in radial cross-section, also defining a substantially crescent-shaped geometry having a greatest radius thereof at a palmer-most radius of said solid body; and an outer circumferential strap having an axial width proportioned to that of said inner strap and having a segment thereof secured to an opposing anterior segment of said inner strap, said outer strap further having a palmer segment adhered along an entirety of an inner side thereof to of a palmer surface of said crescent-shaped solid body.

In view of the above, it is a general object of the invention to provide a wrist support for the reduction of stress, strain and pain associated with repetitive and long term manipulation of industrial devices inclusive of computer input means.

Another object is to provide a portable wrist support device including a wrist support pad positioned exclusively within the wrist region of the palmer side thereof and which is naturally deformable to the contour of the user's wrist.

It is a further object of the invention to provide a device of the above type that may be worn throughout the work day without requirement for removal of the same during segments of the day not associated with manipulation of computer input means such as a keyboard or mouse.

It is still further object to provide a portable wrist support of the above type such that a user's fingers, hand and wrist retain freedom to move within their natural or desired range of motion.

It is a still additional object to provide a portable wrist support means of the above type which is machine-washable (and therefore more hygienic), simple to manufacture, compact, lightweight and inexpensive.

The above and yet other objects and advantages of the present invention will become apparent from the hereinafter set forth Brief Description of the Drawings and Detailed Description of the Invention.

### BRIEF DESCRIPTION OF THE DRAWINGS

FIG. 1 is a view of a portable wrist support device in accordance with the present invention affixed to a wrist of a user.

FIG. 2 is a radial side perspective view of the invention in which a fabric covering of a cross-sectional surface thereof has been removed for purposes of explanation of the invention.

FIG. 3 is a vertical cross-sectional view taken along Line 3-3 of FIG. 2.

FIG. 4 is an operational view showing use of the invention in association with a computer mouse.

FIG. 5 is an operational view showing use of the invention with a computer keyboard.

FIG. 6 is a perspective view of a second embodiment of the invention.

### DETAILED DESCRIPTION OF THE INVENTION

With reference to the perspective view of FIG. 1, the inventive device 10 may be seen positioned upon the wrist 12 of a user thereof.

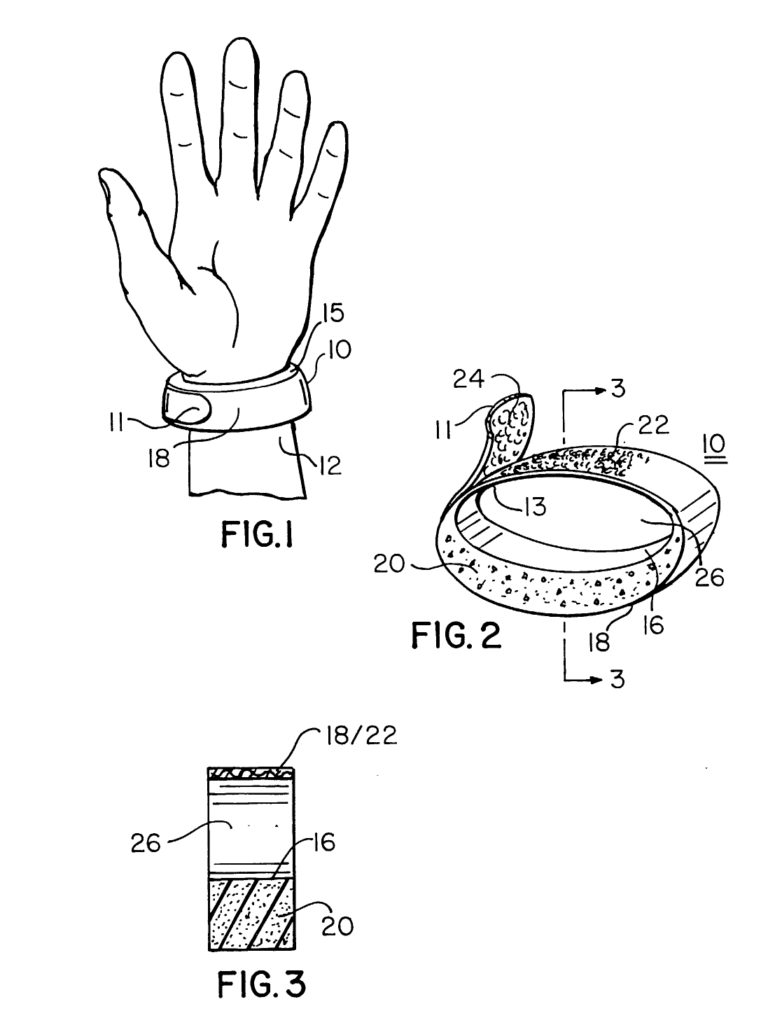

The device may be seen to also include a first end 11 and a second end 13 (see also FIG. 2), an inner surface 16, an outer surface 18, and an axial surface covered by fabric 15.

As may be noted in the perspective view of FIG. 2, fabric 15 has been removed for purposes of description of the invention. The basic geometry thereof may be seen to include a crescent-shaped cross portion 20 which is disposed upon the lower or palmer side of the wrist when the device is secured to the user 12. Accordingly, positioned between inner surface 16 and outer surface 18 is a light compressible material which may be selected from a group of material such as a high-density foam, sponge, dried or organic husks, down or feathers, or a resilient gel. See also axial cross-sectional view of FIG. 3 for the relative axial and radial dimensions of portion 20 along the axis of Line 3-3. Accordingly, through the views of FIGS. 2 and 3, it may be appreciated that when ends 11 and 13 are closed through the use of opposing VELCRO surfaces 24 and 22 respectively, an ellipse 26 is formed having a degree of firmness upon the wrist of the user which is comfortable.

The progressive increase in radial cross section of the high-density foam-like material of portion 20 as a function of position toward the vertical axis (corresponding to the cross section of FIG. 3) is designed to maximize the capacity of the device to absorb vibrations otherwise impacting the fingers, wrist, hand and forearm at the anatomical point where their effect is most noticeable, that is, the bottom portion of the wrist. Accordingly, the use of a concave or crescent shape cross-section, of the type shown in FIG. 2, has been found to provide an effective degree of support to the user without requirement for undue quantities of a cushioning material, as is reflected in the above reference to Beh and without the use of awkward elements which must be physically attached to either or both the hand or forearm of the user.

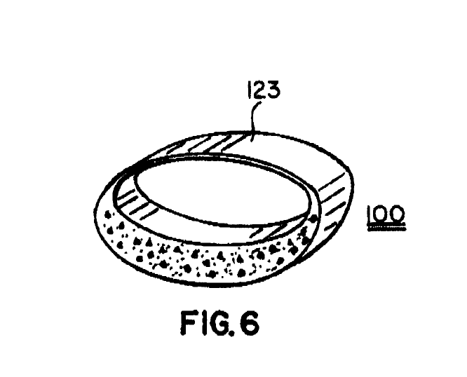

In another embodiment 100 of the invention (see FIG. 6) said first and second ends 11 and 13 respectively, i.e., the anterior portion of the device, may be replaced by a continuous elastomeric strip 123 of like width to that of the first anterior part of the embodiment, to thereby enable a slip-fit or stretchable securement of the device to the wrist.

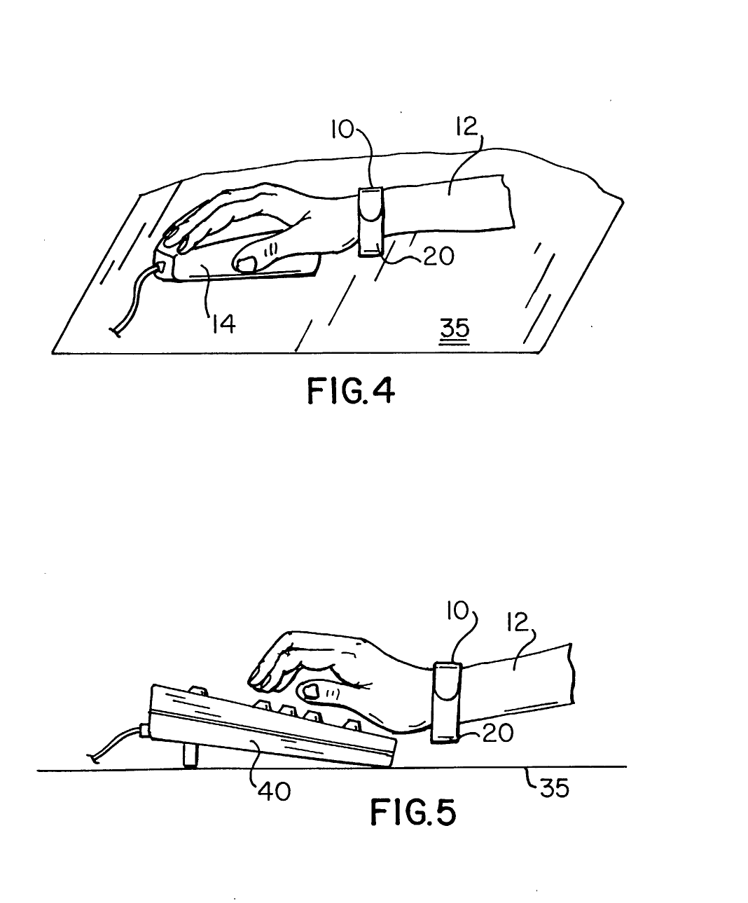

The ease of use of the instant device 10 with a computer mouse 30 is shown in the perspective view of FIG. 4. As may be noted, the high-density foam-like portion 20 of the device 10 may easily rest upon a surface 35 while the user 12 may readily utilize mouse 30 without any form of interference with other or other anatomical functions of the hand, wrist, or forearm. Accordingly, the user is able to alternate between inputting using mouse 30, or keyboard 40 (see FIG. 5), while pursuing to other types of office or household activity.

It is appreciated that in a further embodiment of the invention, the high density foam portion 20 of the invention may be replaced by a bladder such that it may be inflated with compressed air or filled with a liquid or gel to better accommodate a particular height comfortable to particular user. Accordingly, high-density foam portion 20 may be viewed as but one equivalent of many that can serve a comparable purpose within the context of the present invention.

In yet other embodiments of the invention, non functional surfaces thereof may be provided with pockets for the containing of money and keys, may be provided in a variety of colors and design patterns and may incorporate logos, characters, marketing slogans and the like. Further, this fabric is of side surface (see FIG. 1) may in a given embodiment be detachable such that the foam-like or other interior portion may be selectably replaced while washing or cleaning of an outer fabric portion thereof is accomplished.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth and claimed herewith.

I claim:

1. A removable wrist cuff to provide anatomical support to, and protection of, the wrist during repetitive motions of the hand and fingers, said wrist cuff comprising:
(a) an inner circumferential strap proportioned in axial width, circumferential length and circumferential curvature for securement about the entirety of an adult human wrist;
(b) a solid body of a compressibly resilient material, of substantially like axial width of said inner strap, an anterior surface of said body adhered to a palmer outer surface of said inner strap, said solid body of resilient material in radial cross-section, also defining a substantially crescent-shaped geometry having a greatest radius thereof at a palmer-most radius of said solid body; and
(c) an outer circumferential strap having an axial width proportioned to that of said inner strap and having a segment thereof secured to an opposing anterior segment of said inner strap, said outer strap further having a palmer segment adhered along an entirety of an inner side thereof to of a palmer surface of said crescent-shaped solid body.

2. The wrist cuff as recited in claim 1, in which opposing anterior sides of each of said inner and outer straps comprise hook-and-pile means selectably securable and detachable about and against an anterior side of said wrist of a user.

3. The system as recited in claim 1, in which exterior sides of said inner and outer straps of said cuff both comprise an integral elastomeric segment.

4. The system as recited in claim 1, in which said body of resilient material comprises a high-density foam.

5. The system as recited in claim 1, in which said resilient material comprises a gel.

6. The system as recited in claim 1, in which said resilient material comprises an inflatable bladder.

7. The system as recited in claim 2, in which an anterior side of said outer surface of said cuff includes pocket means.

8. The system as recited in claim 3, in which an anterior side of said outer surface of said cuff included pocket means.

9. The wrist cuff as recited in claim 1, in which said greatest radius of said body of resilient material defines a dimension approximately equal to that of said axial width as said inner and outer straps of said cuff.
